(19) United States
(12) Patent Application Publication
(10) Pub. No.: US 2021/0302314 A1
(43) Pub. Date: Sep. 30, 2021

(54) LIGHT DETECTION MODULE AND LIGHT DETECTION DEVICE

(71) Applicant: CITIZEN WATCH CO., LTD., Tokyo (JP)

(72) Inventors: Takahiro KAWADA, Tokyo (JP); Natsuko KITADA, Saitama (JP); Takaaki TAKEISHI, Tokyo (JP); Takaaki NOZAKI, Saitama (JP)

(21) Appl. No.: 17/268,730

(22) PCT Filed: Aug. 15, 2019
(86) PCT No.: PCT/JP2019/032066

§ 371 Date: Feb. 16, 2021

(30) Foreign Application Priority Data
Aug. 27, 2018 (JP) 2018-158207

(51) Int. Cl. G01N 21/64

(52) U.S. Cl. CPC G01N 21/645; A61B 5/0088; G01N 2021/6463

(57) ABSTRACT

A light detection module having a light source emitting excitation lights to a sample a beam splitter reflecting the excitation lights toward an input and output port, and transmitting inspection lights the sample via the input and output port a light receiving element receiving the inspection lights and a housing containing the beam splitter and having a first opening installing the light source, and propagating the excitation lights a second opening corresponding to the input and output port a third opening installing the light receiving element and a fourth opening guiding a stray light transmitted the beam splitter reflecting, wherein the first, second, third and fourth opening communicate with each other at the position of the beam splitter, and the fourth opening extends in a direction perpendicular to the reflecting surface of the beam splitter.

### FIELD

The present invention relates to a light detection module and a light detection device.

### BACKGROUND

In the optical communication field, an optical coupler is known for synthesizing or branching an optical signal using a beam splitter (see, for example, Patent Document 1). In particular, optical devices are known for having a beam splitter (a half mirror, a demultiplexing filter or an optical separation filter), a light source, a light receiver and an optical waveguide, emitting light to the optical waveguide through the beam splitter from the light source, and receiving a light through the beam splitter from the receiver via the same light waveguide (see, for example, Patent Documents 2 to 5).

Further, a light detection device is known for irradiating light to a sample, and receiving (detecting) the inspection light such as fluorescence and a reflected light obtained from the sample in response to the irradiation light. For example, it is described in Patent Document 6 that a dental inspection apparatus detects a fluorescent substance contained in a dental plaque or the like by receiving a response light obtained by irradiating a tooth with light. A fluorescence measurement method is described in Patent Document 7 that irradiates a tooth with excitation light of a specific wavelength, and quantifies an amount of dental plaque or a degree of dental caries by detecting fluorescence emitted by a fluorescent substance.

Related Documents
Patent Document 1 Japanese Laid Open Patent Document No. S58-202423
Patent Document 2 Japanese Laid Open Patent Document No. S61-262711
Patent Document 3 Japanese Laid Open Patent Document No. H6-160674
Patent Document 4 Japanese Laid Open Patent Document No. H8-160259
Patent Document 5 Japanese Laid Open Patent Document No. H8-234061
Patent Document 6 Japanese Laid Open Patent Document No. 2005-324032
Patent Document 7 International Publication No. 2016/140199

### SUMMARY

When a light detection device is realized for performing coaxially irradiation of light to a sample, and detecting an inspection light from the sample by applying the configuration of the above optical couplers, it is important to reduce the influence of a stray light occurring in the beam splitter, in order to increase an detection sensitivity of the light detection device. Further, for example, when an inspection device is applied to dental, it is required to realize a light detection device as a small light detection module. Although a stray light is removed by guiding to a dedicated absorb hole disposed in the housing of the light detection module, if a light detection module is small, it is difficult for the light detection module to provide a stray light absorbing hole having sufficient size, in addition to the optical path of a irradiation light and an inspection light, due to space constraints.

An object of the present invention is to provide a small light detection module performing coaxially irradiation of light to a sample and detection of the inspection light from the sample, with less susceptible to a stray light.

A light detection module having a light source emitting excitation lights to a sample a beam splitter reflecting the excitation lights toward an input and output port, and transmitting inspection lights the sample via the input and output port a light receiving element receiving the inspection lights and a housing containing the beam splitter and having a first opening installing the light source, and propagating the excitation lights a second opening corresponding to the input and output port a third opening installing the light receiving element and a fourth opening guiding a stray light transmitted the beam splitter reflecting, wherein the first, second, third and fourth opening communicate with each other at the position of the beam splitter, and the fourth opening extends in a direction perpendicular to the reflecting surface of the beam splitter.

Further, in the light detection module, it is preferable that the first opening is disposed on the upper surface of the housing, and the second and third openings are disposed on side surfaces facing each other, the beam splitter is fixed to an support member, and the light receiving element is fixed to a light receiving portion substrate, and the support member is deposed in a groove inclined from a position between the ends of the first opening and the third opening sides on a upper surface of the housing, and fixed to the housing with the light receiving portion substrate by a fixture inserted from the side surface of the third opening side.

Further, in the light detection module, it is preferable that the fourth opening is disposed on a lower surface of the housing the lower portion of the housing does not protrude downward and the lower surface of the housing is the same surface as an fourth opening side end portion, the periphery of the fourth opening and the third opening side end portion.

Further, in the light detection module, it is preferable that the inner diameter of the optical path in the third opening is larger than the inner diameter of the optical path of the second opening.

Further, it is preferable that the light detection module further has a light source substrate mounting the light source a lens disposed in the first opening, and condensing the excitation lights and a fixing member fixing the lens, and disposed between the light source substrate and housing, by contacting with the light source substrate and housing, wherein no coating films are disposed, and wiring pattern is exposed on a surface of the light source substrate contacted with the fixing member.

Further, it is preferable that the light detection module further has an other lens disposed in the third opening, and condensing the inspection lights and a circular member fixing the other lens, wherein an opening of the circular member faces the propagation direction of the inspection lights, and functions as an aperture of the inspection lights.

A light detection device having the light detection module according to any one of claims 1 to 6 an optical fiber connected with the input and output port a circuit board driving the light source, and detecting an intensity of the inspection lights received by the light receiving element and a body case containing the light detection module and the circuit board.

Further, it is preferable that the light detection device further has a light emitting portion status displaying for a user, wherein the fourth opening and the light emitting portion are respectively disposed on surfaces facing each other of the body case.

Further, in the light detection device, it is preferable that the light source has a first light emitting element emitting a light including a first wavelength, and a second light emitting element emitting a light including a second wavelength, a first control circuit for detecting the intensity of the inspection lights when the light including the first wavelength is irradiated is disposed on the upper surface of the circuit board, and a second control circuit for detecting the intensity of the inspection lights when the light including the second wavelength is irradiated is disposed on the lower surface of the circuit board, and analog elements included in the first and second control circuits are disposed on one side of the upper and lower surfaces, and digital elements included in the first and second control circuits are disposed on another side of the upper and lower surfaces separately from the analog elements.

The above light detection module is small, and may coaxially perform irradiation of a light to a sample and detection of an inspection light from the sample, with less susceptible to a stray light.

### BRIEF DESCRIPTION OF DRAWINGS

FIG. 1 is a broken perspective view of a fluorescence detecting device 1;

FIG. 2 is a block diagram of a fluorescence detecting device 1;

FIG. 3 is a broken perspective view of the fluorescence detecting module 3;

FIG. 4 is a longitudinal sectional view of the fluorescence detecting module 3;

FIG. 5A is a perspective view of the housing 10;

FIG. 5B is a perspective view of the housing 10;

FIG. 6A is a perspective view of the light source substrate 20;

FIG. 6B is a perspective view of the fixing member 30;

FIG. 7 is a perspective view of a mirror frame 60;

FIG. 8 is a conceptual diagram of light propagating through the optical paths 12b and 12c;

FIG. 9A is explanatory views of the circuit configuration of the fluorescence detecting device 1;

FIG. 9B is explanatory views of the circuit configuration of the fluorescence detecting device 1; and

FIG. 9C is explanatory views of the circuit configuration of the fluorescence detecting device 1.

### DESCRIPTION OF EMBODIMENTS

Hereinafter, with reference to the figures, the light detection module and the light detection device will be described in detail. However, it is to be understood that the invention is not limited to the figures or the following embodiments.

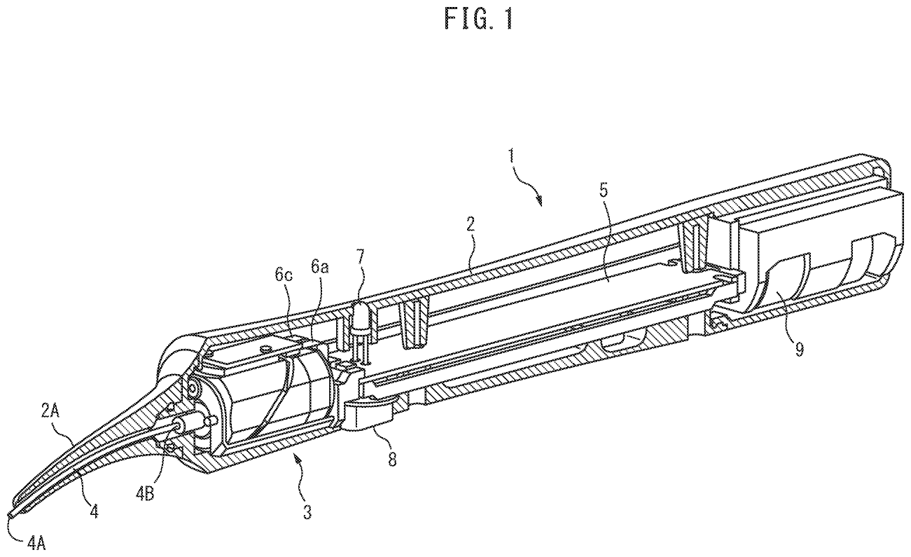

FIG. 1 is a broken perspective view of a fluorescence detecting device 1. The fluorescence detecting device 1 has a body case 2, a fluorescence detecting module 3, a probe fiber 4, a circuit board 5, FPCs (flexible printed wiring board) 6a and 6c, a status display LED (light emitting diode) 7, an operation switch 8 and the battery 9. The fluorescence detecting device 1 is an example of a light detection device, irradiates a excitation light toward a sample from the head 2A of the body case 2, receives a fluorescence (inspection light) generated in the sample in response to a light from the head 2A and detects the fluorescent material of the sample. For example, the fluorescence detecting device 1 may be used as a dental inspection device for detecting protoporphyrin IX contained in a dental plaque as a fluorescent substance.

The body case 2 is a case made of, for example, resin, and contains other components of the fluorescence detecting device 1 such as a fluorescence detecting module 3 and the circuit board 5. The body case 2 is rod-shaped as a whole so that a user may easily hold by his hand, and in the illustrated embodiment, the head 2A directing to a sample is tapered.

The fluorescence detecting module 3 is an example of a light detection module having a light source and a light receiving element to be described later, and is disposed on the head 2A of the body case 2. The fluorescence detecting module 3 detects a small amount of fluorescence from a sample (e.g., fluorescence from a fluorescent material of a plaque) with a high sensitivity by irradiating with an excitation light toward the sample through the probe fiber 4, and receiving the fluorescence incident through the probe fiber 4 occurred in the sample.

The probe fiber 4 is an optical fiber embedded in the head 2A of the body case 2, and is used as a waveguide of an excitation light emitted from the fluorescence detecting module 3 and fluorescence incident on the fluorescence detecting module 3. A head 4A of the probe fiber 4 is open and is directed to a sample during the usage of the fluorescence detecting device 1. A back 4B of the probe fiber 4 is connected to the fluorescence detecting module 3. Although the head 2A of the body case 2 including the probe fiber 4 in the illustrated embodiment is gradually curved, the probe fiber 4 may straightly extend.

The circuit board 5 has a control circuit used for driving a light source of the fluorescence detecting module 3, and detecting an intensity of fluorescence received by light receiving elements. The circuit board 5 has an elongated rectangular shape along the longitudinal direction of the body case 2, and is disposed between the fluorescence detecting module 3 and the battery 9 in the body case 2. The FPC 6a is a substrate for electrically connecting a light source of the fluorescence detecting module 3 with the circuit board 5, and the FPC 6c is a substrate for electrically connecting light receiving elements of the fluorescence detecting module 3 with the circuit board 5.

The status display LED 7 is an example of a light emitting portion, and is disposed on the upper side (front side) of the body case 2 so that a user may easily see a light emitting area. The status display LED 7 is lighting or flushing in order to notify states of the fluorescence detecting device 1 to a user. The operation switch 8 is used by a user when the power supply and irradiation of the excitation light of the fluorescence detecting device 1 are turned on and off, and although the operation switch 8 is disposed on the lower side (back side) of the body case 2 in the illustrated example, the operation switch 8 may be disposed on the upper side of the body case 2. The battery 9 is disposed at an end opposite to the head 2A of the body case 2, and supplies power to the circuit board 5.

As shown in FIG. 1, the probe fiber 4, the fluorescence detecting module 3, the circuit board 5 and the battery 9 are disposed in the above order along the longitudinal direction of the body case 2. The fluorescence detecting module 3 and battery 9 are relatively heavy among the components of the fluorescence detecting device 1, since the fluorescence detecting module 3 and battery 9 are disposed at both ends in the longitudinal direction of the body case 2, the barycenter of the fluorescence detecting device 1 is near the center of the longitudinal direction of the body case 2. Thus, the fluorescence detecting device 1 is well balanced in weight, and therefore a user may easily hold the fluorescence detecting device 1 by his hand.

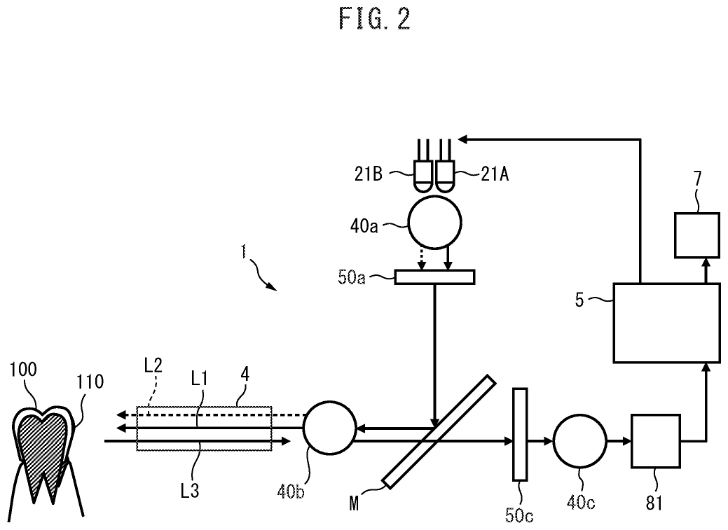

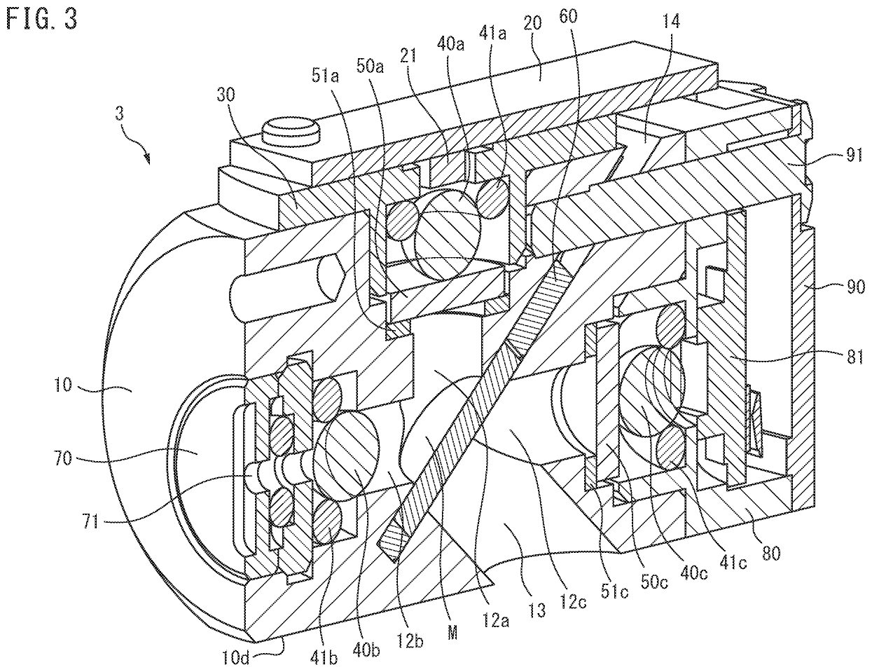

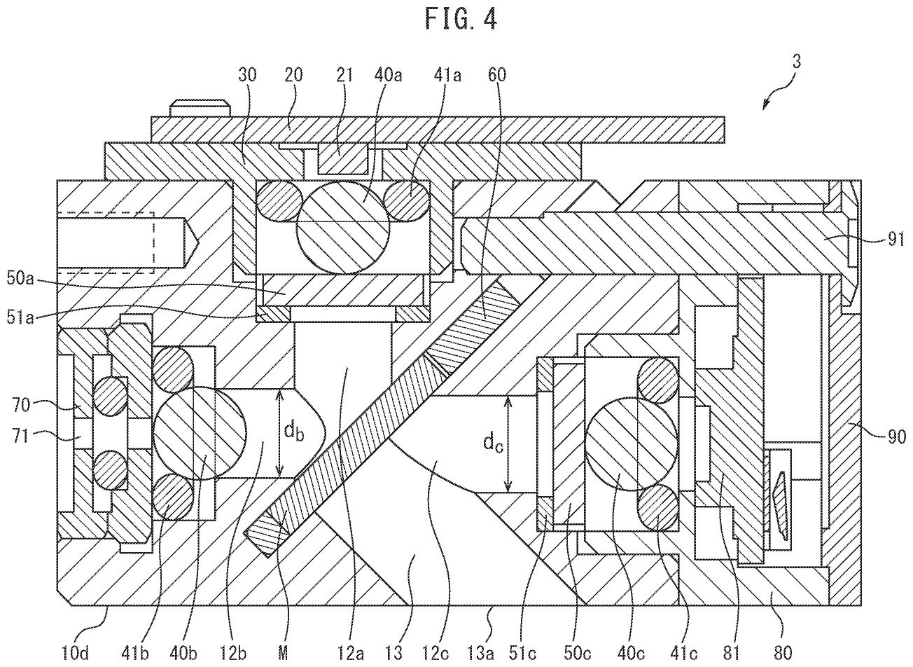

FIG. 2 is a block diagram of a fluorescence detecting device 1. FIG. 3 is a broken perspective view of the fluorescence detecting module 3. FIG. 4 is a longitudinal sectional view of the fluorescence detecting module 3. As shown in FIGS. 3 and 4, the fluorescence detecting module 3 has a housing 10, a light source substrate 20, a fixing member 30, ball lenses 40a to 40c, optical filters 50a and 50c, a mirror frame 60, a fixing member 70, a light receiving portion substrate 80 and a cover 90. A LED package 21 is attached to the light source substrate 20, a mirror M is attached to the mirror frame 60, and a PD (photodiode) element 81 is attached to the light receiving portion substrate 80, respectively. In FIG. 2, it illustrates only components necessary for the description of the function of the fluorescence detecting device 1, among the above components.

As shown in FIG. 2, the fluorescence detecting device 1 has two LED elements 21A and 21B, as the LED package 21 in FIGS. 3 and 4. The LED element 21A is an example of a first light emitting element, and emits a light including a first wavelength having a high excitation efficiency for a detected fluorescent material as an excitation light L1. The LED element 21B is an example of a second light emitting element, and emits a light including a second wavelength longer than the first wavelength as the excitation light L2, and an excitation efficiency of the second wavelength is lower than that of the first wavelength or approximately zero. For example, when a detected target is a plaque phosphor, the first wavelength is preferably in a range of 350 to 430 nm, the second wavelength is preferably in a range of 435 to 500 nm, the LED element 21A may be a violet LED element having a peak wavelength of 405 nm, and the LED element 21B may be a blue LED element having a peak wavelength of 465 nm.

Excitation lights (irradiation lights) L1 and L2 from the LED elements 21A and 21B are incident on a mirror M via the ball lens 40a and the optical filter 50a. The mirror M is formed by a dichroic mirror or a half mirror, and reflects a light in wavelength regions of the excitation lights L1 and L2, and transmits a light in a wavelength region of the fluorescence (inspection light) L3 from a sample. Therefore, the excitation lights L1 and L2 are reflected by the mirror M, and are irradiated to a teeth 100 having, for example, a plaque attachment portion 110 through the probe fiber 4, after condensed by the ball lens 40b. Thus, a fluorescent material contained in a plaque of the plaque attachment portion 110 is excited to generate fluorescent L3 having a peak wavelength around 635 nm and 675 nm. A portion of the fluorescent L3 is incident on the ball lens 40b through the probe fiber 4, transmits the mirror M, and reaches a PD element 81 via the optical filter 50c and the ball lens 40c.

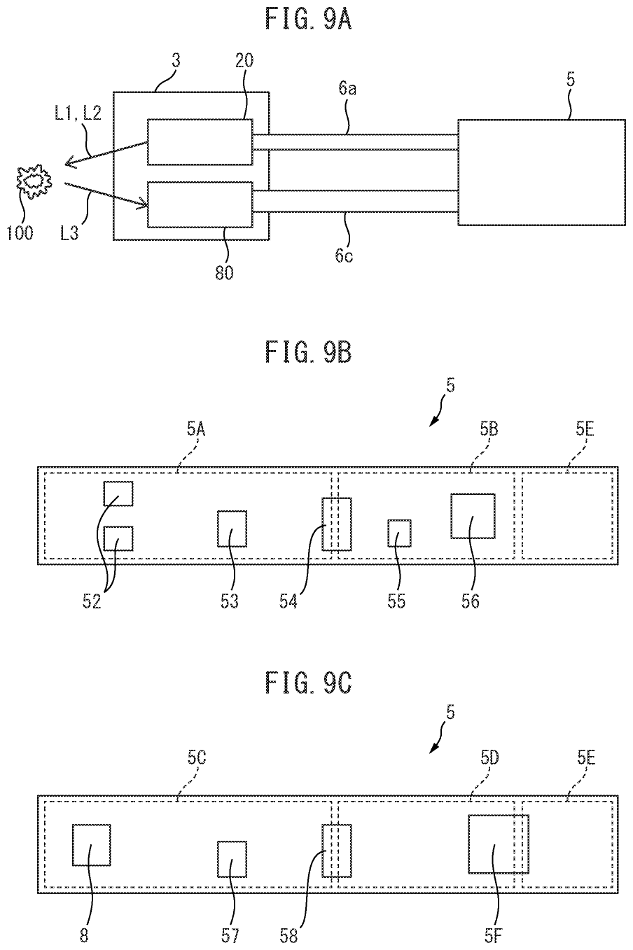

Fluorescence received by the PD element 81 is output to the circuit board 5 after converted into a photocurrent, the presence or absence of a fluorescent material and the amount of a fluorescent material is determined by signal processing of a control circuit disposed on the circuit board 5. The result of the signal processing is notified to a user, for example, by a light of a status display LED 7 or a sound of a built-in buzzer (buzzer 5F in FIG. 9C to be described later). The circuit board 5 alternately irradiates the excitation lights L1 and L2 having wavelengths different from each other to a sample, detects intensities of the fluorescence L3 when the excitation lights L1 and L2 is irradiated respectively, and detects a fluorescent material in the sample based on a ratio or difference of the fluorescent material, for example, by using a fluorescence measurement method described in International Publication No. 2016/140199.

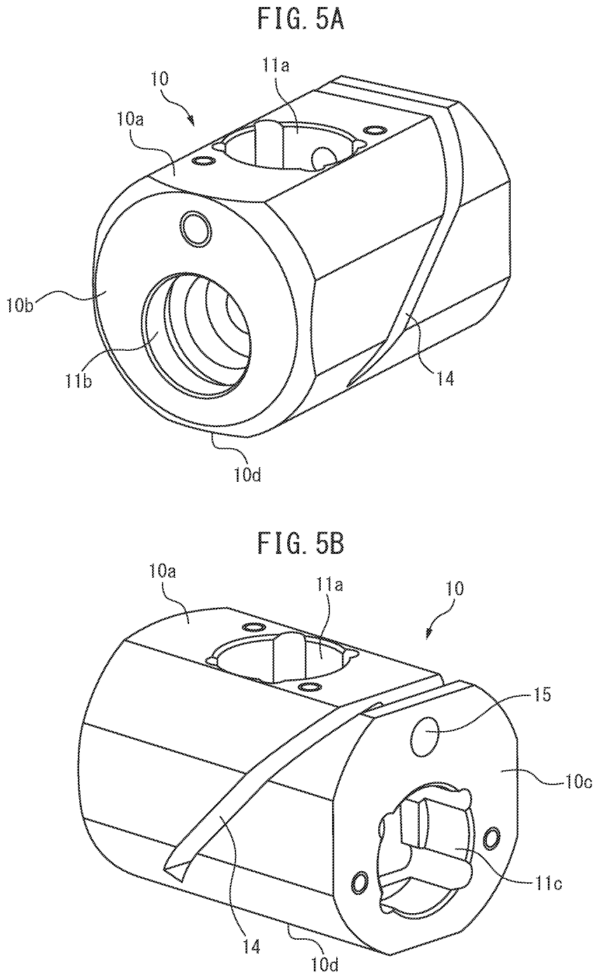

FIGS. 5A and 5B are a perspective view of the housing 10. For example, the housing 10 is an aluminum member anodizing on the whole, and a black aluminum oxide film is formed on the surface of the housing 10, the width and height of the housing 10 are about 1.5 cm, and the depth of the housing 10 is about 2.5 cm. The housing 10 has openings 11a to 11c, a stray light absorbing hole 13, a groove 14 and a threaded hole 15. The openings 11a to 11c, the stray light absorbing hole 13 and the groove 14 communicate with each other at the position of the mirror M, and the groove 14 and the screw hole 15 communicate with each other near the upper surface of the housing 10.

The opening 11a is an example of a first opening, is disposed on the upper surface 10a of the housing 10, and the LED package 21 is installed in the opening 11a as shown in FIGS. 3 and 4. The opening 11b is an example of a second opening, corresponds to a light input and output port of the fluorescence detecting module 3, and is disposed on a side surface 10b of the front of the housing 10 (the head 2A of the body case 2). The opening 11b is connected with the back 4B of the probe fiber 4 shown in FIG. 1. The opening 11c is an example of a third opening, and is disposed on the side surface 10c of the rear of the housing 10 (opposite to the head 2A), and the PD-element 81 is installed in the opening 11c.

The inside of the opening 11a is a light source side optical path 12a propagating the excitation lights L1 and L2. The inside of the opening 11b is a fiber side optical path 12b propagating the excitation lights L1 and L2 reflected by the mirror M and the fluorescence L3 incident from the probe fiber 4. The inside of the opening 11c is a light receiving side optical path 12c propagating the fluorescence L3 transmitted the mirror M. Black films formed on inner walls of the optical paths 12b and 12c by anodizing are removed by polishing, and the inner walls of the optical paths 12b and 12c are light reflective mirror surfaces, so as to detect the fluorescence L3 from a sample, even if the fluorescence L3 is weak. In contrast, no mirror processing is not performed on an inner wall of the optical path 12a, and therefore the light-absorbing black surface formed by the anodizing remains on the inner wall of the optical path 12a. Only a light in the vertical direction toward the mirror M directly from the LED package 21 is passed, and a light in the oblique direction reflected in an irregular direction by the mirror M is removed by absorption at the inner wall of the optical path 12a.

The stray light absorbing hole 13 having a light absorbing inner wall is an example of a fourth opening, and is disposed on a lower surface 10d of the housing 10. A light transmitted the mirror M included in the excitation lights L1 and L2 is guided to the stray light absorbing hole 13, the transmitted light is absorbed by repeatedly reflected by the inner wall of the stray light absorbing hole 13. The stray light absorbing hole 13 is disposed to reliably remove stray light, since if the bottom surface of the inner space of the housing 10 is a black wall surface without holes, a stray light is not completely absorbed by the inner wall surface and becomes a source of noise. The diameter of the stray light absorbing hole 13 may be increased as much as possible in order to enhance the effect of light absorption, and the size of the stray light absorbing hole 13 is larger than the diameters of the optical paths 12a to12c. The light absorbing inner wall of the housing 10 including the stray light absorbing hole 13 may be formed for example, by a non-reflective coating agent such as black nickel plating or a black resin instead of anodizing.

As shown in FIG. 4, the stray light absorbing hole 13 extends in a direction perpendicular to the reflecting surface of the mirror M, and is inclined 45 degrees with respect to the propagation direction of lights in the optical paths 12a, 12b and 12c. If the stray light absorbing hole 13 formed in the vertical direction (on the extension of the optical path 12a), it is not preferable that a portion of the optical paths 12b and 12c may be replaced with the stray light absorbing hole 13. Since the stray light absorbing hole 13 is inclined with respect to a propagation direction of a light, even if a diameter of the stray light absorbing hole 13 is large, a replaced portions of optical paths 12b and 12c are decreased as compared with a case in which a stray light absorbing hole is formed in the vertical direction with respect to the propagation direction. Further, the lengthen of the stray light absorbing hole 13 may be long by inclining the stray light absorbing hole 13 with respect to the propagation direction, even if the housing 10 is small as compared with a case in which a stray light absorbing hole is formed in the vertical direction with respect to the propagation direction. Furthermore, since the opening 13a of the stray light absorbing hole 13 is widened, and therefore a stray light may escape from the fluorescence detecting module 3 to the outside, noise is reduced, by inclining the stray light absorbing hole 13 with respect to the propagation direction, as compared with a case in which a stray light absorbing hole is formed in the vertical direction with respect to the propagation direction. For the above reasons, the stray light absorbing hole 13 is formed at right angles to the mirror M in the fluorescence detecting module 3.

As shown in FIGS. 5A and 5B, the housing 10 has a substantially octagonal columnar shape, and the projecting portion is not formed on the surface of the housing 10. In particular, the lower portion of the housing 10 including a portion around the stray light absorbing hole 13 does not protrude downward, and a lower surface 10d of the housing 10 is the same surface as an opening 11a side end portion, the periphery of the stray light absorbing hole 13 and the opening 11c side end portion as shown in FIGS. 3 and 4. For example, if the housing 10 is molded into a T-shape rather than a columnar shape, and the portion of the stray light absorbing hole 13 is projected downward, the length of the stray light absorbing hole 13 may be long by the length of the T-shape. However, if the housing 10 is molded into a T-shape, the size of the fluorescence detecting module 3 becomes large, and therefore the size of the body case 2 for housing the housing 10 become large, disadvantages such as a difficulty for a user to hold the fluorescence detecting device 1 may occur. Since the stray light absorbing hole 13 is inclined with respect to the vertical direction, and the lower surface 10d is flat, the of light absorption effect may be increased by ensuring the length of the stray light absorbing hole 13, while miniaturizing the housing 10.

The groove 14 is used for positioning with respect to the housing 10 by inserting a mirror frame 60, and is inclined with respect to the vertical direction from a position between the ends of the opening 11a and the opening 11c sides on a upper surface of the housing 10. The screw hole 15 is used for a screw 91 shown in FIGS. 3 and 4. Although the stray light absorbing holes 13 and the groove 14 among the openings 11a to 11c, the stray light absorbing hole 13, the groove 14 and the screw hole 15 are in an open state in the fluorescence detecting module 3, lids (covers) may be disposed for closing the stray light absorbing holes 13 and the groove 14. However, since the stray light absorbing holes 13 and the groove 14 are closed by covered with an inner wall of the body case 2 in a finished product of the fluorescence detecting device 1, there is no particular problem even if the stray light absorbing holes 13 and the groove 14 are an open state in the fluorescence detecting module 3, it is preferable to leave the stray light absorbing holes 13 and the groove 14 in the open state, since the number of parts is reduced, and therefore the manufacturing cost is reduced.

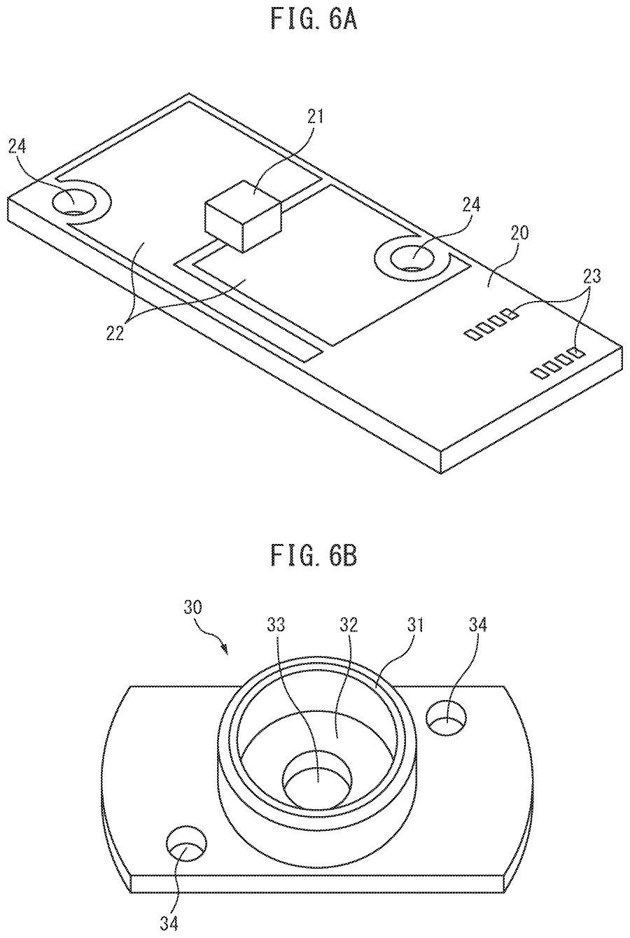

FIG. 6A is a perspective view of the light source substrate 20. The light source substrate 20 is a substrate mounting the LED package 21 that is a light source of the fluorescence detecting module 3 (fluorescence detecting device 1), and has the wiring pattern 22, a connection terminal 23 and two screw holes 24. The light source substrate 20 is attached to the upper surface side of the housing 10 via the fixing member 30, as shown in FIGS. 3 and 4. The upper surface of the light source substrate 20 shown in FIG. 6A faces downward (lower side in FIGS. 3 and 4) in a state attached to the housing 10. Two LED elements 21A and 21B shown in FIG. 2 are included in The LED package 21 as one package, and the LED elements 21A and 21B emit respectively excitation lights L1 and L2 having wavelengths different from each other (e.g., 405 nm and 465 nm). The light source of the fluorescence detecting module 3 is not limited to the LED elements, for example, may be semiconductor lasers.

The wiring pattern 22 and the connection terminal 23 are used for supplying power to the LED package 21, and are formed on the upper surface of the light source substrate 20. The connecting terminal 23 is connected to the FPC 6a shown in FIG. 1. The screw holes 24 are used for screwing the light source substrate 20 with respect to the housing 10, and are formed one by one at a corner facing the diagonal direction of the light source substrate 20.

FIG. 6B is a perspective view of the fixing member 30. The fixing member 30 is a member used for fixing the ball lens 40a disposed in the opening 11a of the light source side, made of a material having well heat dissipation characteristics (e.g., metal such as aluminum), and has an circular wall portion 31, an through hole 33 and two screw holes 34. The circular wall portion 31 is disposed in the central of the upper surface shown in FIG. GB, a ball lens 40a and an O-ring 41a for receiving the ball lens 40a are disposed in the circular space 32 surrounded by the circular wall portion 31. The fixing member 30 is attached to the upper surface of the housing 10 upside down so that the circular wall portion 31 fits in the opening 11a and covers the opening 11a. The through hole 33 is formed in the central of the area surrounded by the circular wall portion 31, and the LED package 21 is disposed in the through hole 33, as shown in FIGS. 3 and 4.

The screw holes 34 are formed one by one in a corner portion facing the diagonal direction of the fixing member 30 in the same positional relationship as the two screw holes 24 of the light source substrate 20. The light source substrate 20 and the fixing member 30 are fixed to the housing 10, by aligning the position of the screw holes 24 and 34 and inserting screws in the screw holes 24 and 34. Thus, the fixing member 30 is used for fixing the ball lens 40a and the O-ring 41a, and fixing the light source substrate 20.

No resists (coating films) are disposed on a mounting surface of the LED package 21 in the light source substrate 20 (contact surface with the fixing member 30), and the mounting surface is a metal surface exposing the wiring pattern 22. Heat generated by the light emission of the LED package 21 are easily radiated to the metal housing 10 side, since the mounting surface is a metal surface. Since the fixing member 30 is disposed between the light source substrate 20 and the housing 10 in contact therewith, the mounting surface of the light source substrate 20 is in contact with the fixing member 30, and the fixing member 30 is in contact with the housing 10. Since the housing 10 and the fixing member 30 are formed so as to isolate each other, no problems occur even if the wiring pattern 22 and the fixing member 30 is directly contacted, and heat is radiated through a path including the mounting surface of the light source substrate 20, the fixing member 30 and the housing 10 by such an arrangement. The fixing member 30 also is used as a heat dissipation path of the LED package 21.

Regarding heat radiation of LED packages, it is general that heat is radiated from a back side of a mounting substrate. However, if the above structure is applied to the fluorescence detecting module 3, it is necessary to provide a contact between the rear surface of the light source substrate 20 which is the upper side in FIGS. 3 and 4 and the body case 2, and therefore extra structures for heat dissipation are required. Heat dissipation structures are simplified, and the fluorescence detecting module 3 is downsized, in addition to easy heat dissipation by utilizing the fixing member 30 integrated with the housing 10 as a heat dissipation path. Even if the housing 10 is formed of a material other than metal such as resin, if the fixing member 30 is formed of a metal having well heat dissipation characteristics such as aluminum or copper, the fixing member 30 functions as heat dissipation path. Further, a material of the fixing member 30 is necessarily a metal as long as the material has a high thermal conductivity, for example, a resin including heat conductive fillers such as inorganic particles (high thermal conductivity resin).

The ball lens 40a (lens) is disposed in the opening 11a fixed in a circular space 32 of the fixing member 30, and condenses the excitation lights L1 and L2 emitted from the LED package 21. The ball lens 40b is disposed in the opening 11b, and condenses the excitation lights L1 and L2 reflected by the mirror M and incident on the probe fiber 4 and the fluorescence L3 incident in the optical path 12b from the probe fiber 4. The ball lens 40c (other lens) is disposed immediately before the PD element 81 in the opening 11c, and condense the fluorescent light L3 transmitted through the mirror M. Although all of the ball lenses 40a to 40c have a spherical shape and similar size, the shape and size of the ball lenses are not limited thereto. For example, convex lenses may be used instead of ball lenses 40a to 40c.

As shown in FIGS. 3 and 4, the ball lenses 40a to 40c are respectively fixed by rubber O-rings 41a to 41c. The 0-rings 41a to 41c have an opening in the center, since the O-rings 41a to 41c are circular members, and all of openings face the propagation direction of the excitation lights L1 and L2 or fluorescence L3. Thus, the O-rings 41a to 41c are lens receivers of the ball lens 40a to 40c, respectively, and function as an aperture of the excitation lights L1 and L2 or the fluorescent light L3 incident on the ball lenses 40a to 40c. In particular, since the fluorescence L3 is scattered light, it is desirable to narrow down the fluorescence L3 in front of the PD element 81, the O-ring 41c is used as diaphragms, and therefore it is not necessary to place diaphragms as separate members in the fluorescence detecting module 3. The above structure has an advantageous effect in terms of downsizing of the fluorescence detecting module 3, and reducing the number of parts and manufacturing cost.

The optical filter 50a is a filter for transmitting the excitation lights L1 and L2 and cutting the light in the wavelength range of the fluorescence L3. For example, when the peak wavelengths of the excitation lights L1 and L2 are 405 nm and 465 nm and a plaque is detected, since a wavelength range of the plaque-derived fluorescence L3 is about 620 to 690 nm, it is preferable to use an optical filter as the optical filter 50a cutting a light having a wavelength of 500 nm or more. The optical filter 50a is sandwiched between a buffer rubber 51a and the ball lens 40a having an empty hole in the center so that a light may be transmitted, and the optical filter 50a is fixed immediately below the ball lens 40a in the opening 11a.

The optical filter 50c is a filter for cutting a light having a wavelength range other than the fluorescence L3. When a plaque is detected, a filter may be used as the optical filter 50c to cut a light in a wavelength range except for 620 to 690 nm. The optical filter 50c is sandwiched between a buffer rubber 51c and the ball lens 40c having a hole in the center, and the optical filter 50c is fixed to the position of the opening 11b side than the ball lens 40c in the opening 11c. Since the ball lenses 40a and 40c and the optical filters 50a and 50c are respectively sandwiched between the rubber O-rings 41a and 41c and the buffer rubbers 51a and 51c, impact resistances of the ball lenses 40a and 40c and the optical filters 50a and 50c are improved.

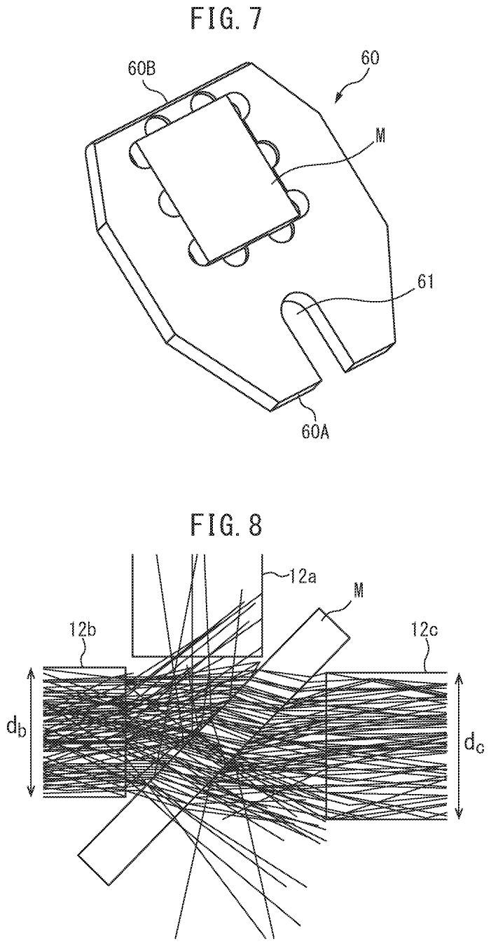

FIG. 7 is a perspective view of a mirror frame 60. The mirror frame 60 is a frame (support member) fixing the mirror M, and is inserted into the groove 14 of the housing 10 so as to arrange an end portion 60A shown in FIG. 7 toward the upper side and an end portion 60B toward the lower side. A recess 61 is formed in the end portion 60A, and is a screw groove for passing a screw 91 shown in FIGS. 3 and 4. The mirror M is an example of a beam splitter, is formed by a dichroic mirror or a half mirror, and is adhered to a position close to the end 60B of the mirror frame 60 at a lower side in the housing 10. The mirror M reflects the majority of the excitation lights L1 and L2 propagating through the optical path 12a toward the optical path 12b, and transmits the fluorescence L3 incident through the optical path 12b. For example, a prism type mirror may be used as the beam splitter, instead of the mirror M of the planar type mirror, and the shape of the mirror M is not limited.

The fixing member 70 shown in FIGS. 3 and 4 is a member for pressing the O-ring 41b to fix the ball lens 40b, and is attached so as to cover the opening 11b. The fixing member 70 has an opening 71 in the central, and the back 4B of the probe fiber 4 shown in FIG. 1 is fixed to the opening 71.

The light receiving portion substrate 80 is a substrate mounting a PD element 81, similar to the fixing member 30, has an circular wall portion in which the ball lens 40c and the O-ring 41c are disposed in a circular space therein, and the circular wall portion fits in the opening 11c is attached to the side surface of the housing 10 so as to cover the opening 11c. The PD element 81 is fixed to a position on the extension line of the optical path 12c in the light receiving portion substrate 80. The PD element 81 is an example of a light receiving element, receives the fluorescent light L3 transmitted through the optical filter 50c and the ball lens 40c, and outputs a photocurrent corresponding to an intensity of the fluorescent light L3 to the circuit board 5.

The cover 90 is a member for covering the light receiving portion substrate 80, is attached to the side surface of the opening 11c side of the housing 10, by a screw 91 penetrating the cover 90 and the light receiving portion substrate 80, and is fixed to the housing 10 together with the light receiving portion substrate 80. The screw 91 is an example of a fixture, and has a length in which the screw 91 is inserted into the screw hole 15 as shown in FIG. 5B, the screw 91 extends from the cover 90 to the front of the circular wall portion 31 of the fixing member 30 beyond the groove 14. Thus, the mirror frame 60 is also sandwiched within the groove 14 by screws 91. In the fluorescence detecting module 3, three members of the mirror frame 60, the light receiving portion substrate 80 and the cover 90 are fixed simultaneously by a single screw 91. Since it is not necessary to fix the three members separately with three screws by such arrangements of the mirror frame 60 and the screw 91, the size of the fluorescence detecting module 3 may be reduced.

The fixture may be a screw or pin or the like as long as it may fix the above three members, types of the fixture are not particularly limited.

FIG. 8 is a conceptual diagram of light propagating through the optical paths 12b and 12c. In FIG. 8, the periphery of the mirror M in the housing 10 is enlarged, the fluorescence toward the optical path 12c of the light receiving side from the optical path 12b of the fiber side is shown schematically in a number of solid lines. Since fluorescence from a sample is a diffused light (scattered light), not all of fluorescence propagates in the direction of the optical path 12c, partially reflected or refracted to the stray light absorbing hole 13 side of the light source side of the optical path 12a or opposite side of the optical path 12a, and therefore loss occurs at spaces surrounding the mirror M. Thus, the inner diameter dc of the optical path 12c is larger than the inner diameter db of the optical path 12b in the fluorescence detecting module 3. Since the inner diameter dc is larger than the inner diameter db, the amount of light received by the PD element 81 increased as compared with a case in which the inner diameters dc and db are similar, and therefore the propagation efficiency of the fluorescence L3 (i.e., the detection sensitivity of the fluorescence detecting device 1) is increased.

If an incident light from a sample is sufficiently narrowed down by the ball lens 40b, the inner diameter dc may not necessarily be larger than the inner diameter db, however if the opening 11a to 11c will be used lenses of different types (sizes), the manufacturing cost is increased. The propagation efficiency of fluorescence may be increased while reducing the types of parts and the manufacturing cost, by using lenses having similar size as the three ball lenses 40a to 40c, and forming the inner diameter dc larger than the inner diameter db.

The stray light absorbing hole 13 is disposed on the lower surface of the housing 10 as shown in FIGS. 3 and 4, and the fluorescence detecting module 3 is installed in the body case 2 of the fluorescence detecting device 1 with the lower surface facing downward. Further, the status display LED 7 is disposed on the upper side of the body case 2 as shown in FIG. 1. The stray light absorbing hole 13 and the status display LED 7 are respectively disposed on surfaces facing each other of the body case 2. If the stray light absorbing hole 13 and the state display LED 7 are placed on the same side of the body case 2, a light from the state display LED 7 enters the stray light absorbing hole 13 and becomes noise, however since both the stray light absorbing hole 13 and the state display LED 7 are disposed in opposite positions each other, the effect on the detection by a light emitted from the state display LED 7 is reduced.

FIGS. 9A to 9C are explanatory views of the circuit configuration of the fluorescence detecting device 1. FIG. 9A schematically shows the connecting relationship among the light source substrate 20, the light receiving portion substrate 80, the FPCs 6a and 6c and the circuit board 5. As shown in FIG. 9A, the FPC 6a electrically connects the light source substrate 20 with the circuit board 5, and the FPC 6c electrically connects the light receiving portion substrate 80 with the circuit board 5. The FPC 6c mounts a current-voltage converting circuit (transimpedance amplifier) for converting the photocurrent from the PD-element 81 to a voltage.

FIGS. 9B and 9C are respectively a schematic top view and a bottom view of the circuit board 5. The circuit elements mounted on the circuit board 5 includes a band-pass filter 52, lock-in amplifiers 53 and 57, A/D converters 54 and 58, a LED drivers 55 and a CPU 56 (microcomputer). The band-pass filter 52 and the lock-in amplifiers 53 and 57 are analog elements, A/D converters 54 and 58 are analog and digital mixed elements, and LED driver 55 and CPU 56 area digital elements.

Since the fluorescence detecting device 1 detects the fluorescence intensity using two phase lock-in amplifiers, the circuit board 5 has the lock-in amplifiers and the A/D converters in the two systems (two sets). Although it is desirable to place analog and digital circuits separately, since the circuit board 5 mounts the analog and digital circuits in two systems, the arrangement becomes complicated if all of the analog and digital circuits mounted on one surface of the substrate. Further, for example, the analog circuits are mounted on the upper surface of the substrate, whereas the digital circuits are mounted on the lower surface of the substrate, the routing of the power supply wires becomes difficult. Therefore, in the circuit board 5, the lock-in amplifiers and the A/D converters in the two systems are disposed one by one on the upper and the lower surfaces. A first control circuit for detecting the intensity of the fluorescence L3 when the excitation light L1 is irradiated is disposed on the upper surface of the circuit board 5, and a second control circuit for detecting the intensity of the fluorescence L3 when the excitation light L2 is irradiated is disposed on the lower surface of the circuit board 5.

Then, in the circuit board 5, analog elements including the band-pass filter 52 and the lock-in amplifiers 53 and 57 are disposed on the left side in the figure close to the fluorescence detecting module 3, digital elements including the LED driver 55 and CPU 56 are disposed on the right side in the figure close to the battery 9, analog and digital mixed elements including A/D converters 54 and 58 are disposed on the center of the circuit board 5. The analog elements are mounted on one side of the upper and the bottom surfaces of the circuit board 5, the digital elements are mounted on the other side of the upper and the lower surfaces of the circuit board 5 separately from the analog elements. Reference numerals 5A and 5B in FIG. 9B indicate the analog circuit and the digital circuit on the upper surface (first control circuit), reference numerals 5C and 5D in FIG. 9C indicate the analog circuit and the digital circuit on the lower surface (second control circuit), reference numerals 5E indicates a power supply circuit.

The analog circuits may easily separate the digital circuits by such an arrangement, and the area of the substrate is reduced as compared with a case in which all the elements are placed on one side of the substrate, and therefore the downsizing effect may be obtained.

Reference numeral 8 in FIG. 9C indicates an operation switch, reference numeral 5F indicates a buzzer for notifying the state of the fluorescence detecting device 1 such as the presence or absence of detection to a user. Since the buzzer 5F is a noise source, the buzzer 5F is disposed near the power supply circuit 5E and farthest from the analog circuit 5C side at the lower surface of the circuit board 5. Although not shown, the status display LED 7 is disposed between the band-pass filter 52 and the lock-in amplifier 53 in the upper analog-circuit 5A.

As above described, the fluorescence detecting module 3 is small and lightweight, and irradiation of the excitation light and detection of fluorescence may be coaxially performed. When the fluorescence detecting device 1 including the fluorescence detecting module 3 is used as an inspection apparatus for dental use, by inserting the probe fiber 4, for example, visually difficult teeth gap or deep, the weak fluorescence and the change of optical characteristics of fluorescence generated by a sample may be detected at a position away from the sample. Since LEDs having the visible light range are used for the light sources, safety may be ensured and power consumption may be reduced. Since the environmental light may be cut by filtering process, the fluorescence detecting device 1 may be used in a living environment without selecting a detection location. The emission wavelength of the LED package 21 and the characteristics of the optical filters 50a and 50c may be freely selected according to the detection target, and light other than fluorescence such as reflected light may be detected as the inspection light.

1. A light detection module comprising:
a light source emitting excitation lights to a sample;
a beam splitter reflecting the excitation lights toward an input and output port, and transmitting inspection lights the sample via the input and output port;
a light receiving element receiving the inspection lights; and
a housing containing the beam splitter and having:
a first opening installing the light source, and propagating the excitation lights;
a second opening corresponding to the input and output port;
a third opening installing the light receiving element; and
a fourth opening guiding a stray light transmitted the beam splitter reflecting, wherein
the first, second, third and fourth opening communicate with each other at the position of the beam splitter, and
the fourth opening extends in a direction perpendicular to the reflecting surface of the beam splitter.

2. The light detection module according to claim 1, wherein
the first opening is disposed on the upper surface of the housing, and the second and third openings are disposed on side surfaces facing each other,
the beam splitter is fixed to an support member, and the light receiving element is fixed to a light receiving portion substrate, and
the support member is deposed in a groove inclined from a position between the ends of the first opening and the third opening sides on a upper surface of the housing, and fixed to the housing with the light receiving portion substrate by a fixture inserted from the side surface of the third opening side.

3. The light detection module according to claim 2, wherein
the fourth opening is disposed on a lower surface of the housing;
the lower portion of the housing does not protrude downward; and
the lower surface of the housing is the same surface as an fourth opening side end portion, the periphery of the fourth opening and the third opening side end portion.

4. The light detection module according to claim 1, wherein the inner diameter of the optical path in the third opening is larger than the inner diameter of the optical path of the second opening.

5. The light detection module according to claim 1, further comprising:
a light source substrate mounting the light source;
a lens disposed in the first opening, and condensing the excitation lights; and
a fixing member fixing the lens, and disposed between the light source substrate and housing, by contacting with the light source substrate and housing, wherein no coating films are disposed, and wiring pattern is exposed on a surface of the light source substrate contacted with the fixing member.

6. The light detection module according to claim 5, further comprising:
an other lens disposed in the third opening, and condensing the inspection lights; and
a circular member fixing the other lens, wherein an opening of the circular member faces the propagation direction of the inspection lights, and functions as an aperture of the inspection lights.

7. A light detection device comprising:
the light detection module according to claim 1;
an optical fiber connected with the input and output port;
a circuit board driving the light source, and detecting an intensity of the inspection lights received by the light receiving element; and
a body case containing the light detection module and the circuit board.

8. The light detection device according to claim 7, further comprising a light emitting portion status displaying for a user, wherein the fourth opening and the light emitting portion are respectively disposed on surfaces facing each other of the body case.

9. The light detection device according to claim 7, wherein
the light source has a first light emitting element emitting a light including a first wavelength, and a second light emitting element emitting a light including a second wavelength,
a first control circuit for detecting the intensity of the inspection lights when the light including the first wavelength is irradiated is disposed on the upper surface of the circuit board, and a second control circuit for detecting the intensity of the inspection lights when the light including the second wavelength is irradiated is disposed on the lower surface of the circuit board, and
analog elements included in the first and second control circuits are disposed on one side of the upper and lower surfaces, and digital elements included in the first and second control circuits are disposed on another side of the upper and lower surfaces separately from the analog elements.
